(19) United States
(12) Patent Application Publication
(10) Pub. No.: US 2006/0272049 A1
(43) Pub. Date: Nov. 30, 2006

(54) Insect resistance using inhibition of gene expression

(76) Inventors: Peter Michael Waterhouse, O'Connor (AU); Steven Whyard, Higgins (AU); Jeroen Van Rie, Eeklo (BE)

Correspondence Address: BUCHANAN, INGERSOLL & ROONEY PC, POST OFFICE BOX 1404, ALEXANDRIA, VA, 22313-1404, US

(21) Appl. No.: 10/579,503

(22) PCT Filed: Nov. 17, 2004
(86) PCT No.: PCT/EP04/13049

§ 371 Date: May 16, 2006

Related U.S. Application Data
(60) Provisional application No. 60/520,306, filed on Nov. 17, 2003.

(51) Int. Cl. A01H 1/00; C07H 21/04; C12N 15/82; C12N 5/04; C07K 14/415

(52) U.S. Cl. 800/279; 435/419; 435/468; 530/370; 536/23.6

(57) ABSTRACT

The current invention provides methods to silence insect genes by using unpackaged dsRNA or siRNA, in one embodiment such dsRNA or siRNA is present in plant vascular tissue, preferably phloem, more particularly phloem sap, and the insect is a plant sap-sucking insect. Also provided are DNA sequences which when transcribed yield a double-stranded RNA molecule capable of reducing the expression of an essential gene of a plant sap-sucking insect, methods of using such DNA sequences and plants or plant cells transformed with such DNA sequences. Also provided is the use of cationic oligopeptides that facilitate the entry of dsRNA or siRNA molecules in insect cells, such as plant sap-sucking insect cells.

### INTRODUCTION

Several approaches are currently available to obtain plants with increased resistance to plant insect pests. However, for plant sap-sucking insects such as aphids, planthoppers, stinkbugs, and whiteflies, only limited options are currently available to protect plants using transgenic approaches. Plants expressing Bacillus thuringiensis toxins targeted to Lepidopteran insects have not been shown to possess enhanced resistance towards plant sap-sucking insects (Rao et al., 1998).

dsRNA technology has been proven to be highly specific and highly effective in silencing endogenous genes in several organisms. However, the dsRNA provided to date is typically packaged in a bacterial or yeast cell or in transfection promoting agents such as liposomes. In this invention, it has now been shown that naked, unpackaged dsRNA can be used to silence genes in plant sap-sucking insects.

Plant sap-sucking insects typically feed from the sap in the vascular system of plants which they tap into with the stylets of their proboscis, causing a reduction in plant vitality and the spreading of several plant viral diseases. Plant sap-sucking insects typically have a short life-cycle and are capable of building up immense populations very quickly on a host plant.

### BACKGROUND OF THE INVENTION

Published PCT application WO 00/01846 generally refers to a method of alleviating pest infestation of plants, which method comprises a) identifying a DNA sequence from said pest which is critical either for its survival, growth, proliferation or reproduction, b) cloning said sequence or a fragment thereof in a suitable vector relative to one or more promoters capable of transcribing said sequence to RNA or dsRNA upon binding of an appropriate transcription factor to said promoters, and c) introducing said vector into the plant. The plant pests referred to in this published PCT application are nematodes.

U.S. Pat. No. 6,326,193 refers to the use of recombinant insect viruses such as baculoviruses expressing dsRNA to silence selected insect genes.

Published PCT application WO 99/32619 describes generally that dsRNA may be used to reduce crop destruction by other plant pathogens such as arachnids, insects, nematodes, protozoans, bacteria, or fungi. This application shows that E. coli bacteria expressing dsRNAs can confer specific inhibitory effects on C. elegans nematode larvae that feed on these bacteria.

Timmons et al. (2001) describe that ingestion of bacteria expressing dsRNAs can produce genetic interference in the nematode Caenorhabditis elegans. These authors describe that from a bioengineering perspective, it may be possible to use a bioengineered dsRNA comestible to modify gene expression in any organism which has a gene-specific dsRNA response similar to that of C. elegans. To respond to such an intervention, that target organism would need to have both a dsRNA-response mechanism (RNAi) and a dsRNA uptake/spreading mechanism that would produce responses such as those observed in C. elegans.

Similarly, inhibition of gene expression after ingestion of bacteria expressing double-stranded RNA was also reported for freshwater planarian flatworms (Newmark et al., 2003).

Published PCT patent application WO 2004/005485 describes the use of vectors comprising sequences designed to control plant-parasitic nematodes by RNA interference, and transgenic plants transformed with such vectors.

Published US patent application 20030180945 generally describes chimeric genes capable of producing antisense or sense RNA equipped with a prokaryotic promoter suitable for expression of the antisense or sense RNA in a particular prokaryotic host. The prokaryotic host can be used as a source of antisense and/or sense RNA, e.g., by feeding it to an animal, such as a nematode or an insect, in which the silencing of the target gene is envisioned and monitored by reduction of the expression of the reporter gene. The target gene and reporter genes should be genes present in the cells of the target eukaryotic organism, and not in the prokaryotic host organism.

Published US patent application 20030154508 describes a method for pest control comprising exposing said pest to a compound which disrupts, within said pest, a cation-amino acid transporter/channel protein. Generally described are plant cells genetically modified to produce at least one double-stranded RNAi that is designed to be taken up by pests during feeding to block expression (or the function) of a target gene. It is described that RNAi can be used to reduce or prevent message translation in any tissue of the pest because of its ability to cross tissue and cellular boundaries. It is further described that RNAi that is contacted with a pest by soaking, injection, or consumption of a food source will cross tissue and cellular boundaries, and that RNAi can also be used as an epigenetic factor to prevent the proliferation of subsequent generations of pests.

Published PCT patent application WO 01/37654 generally describes dsRNA targeted against piercing-sucking insects and chewing insects, and that double stranded RNAs intended to confer sap-sucking insect resistance would preferentially be expressed in plant tissues on which such insects feed, e.g., primary and secondary phloem elements, and would be taken up by the insect via its sucking mechanism, e.g., its stylet. The only insect for which application of dsRNA is exemplified is Manduca sexta, a Lepidopteran insect. Susceptibility of this insect to RNAi was determined by treating larvae with dsRNA by either feeding or by direct injection. The results from these experiments show that with dsRNA sequences derived from three separate genes, injection into M. sexta leads to substantial decrease in expression of the endogenous gene. No results of the feeding experiment are reported.

Published PCT patent application WO 02/14472 describes methods for inhibiting target gene expression, by expressing in a cell a nucleic acid construct comprising an inverted repeat and a sense or antisense region having substantial sequence identity to a target gene, wherein the inverted repeat is unrelated to the target gene. Listed as one of the possible targets are insects such as sucking insects.

US published patent application 20030150017 describe the use of RNA molecules homologous or complementary to a nucleotide sequence of a plant pest such as nematodes and insects. Application of RNAi to a model insect species, the lepidopteran insect Helicoverpa armigera, in the model plant lettuce, is suggested in the Examples, but no feeding experiment is done and no results are reported.

Published PCT patent application WO 03/004644 A1 describes that the evolutionary distance between nematodes and insects is considerable, and that there is no reason to assume that while feeding dsRNA to C. elegans was successful, it would be a technique easily transferable to insects. It further describes the use of dsRNA technology to arthropods, and shows that direct feeding of naked, unpackaged, dsRNA failed to produce an RNAi phenotype in Drosophila melanogaster and Helicoverpa armigera, indicating that transfection promoting agents such as liposomes were necessary for effective transfection in these species. This application generally envisages that in arthropods with a simple digestive system naked dsRNA may be effective in obtaining gene silencing. No results of the delivery of naked dsRNA to any arthropod are included in this application.

Gura (2003) describes that while in C. elegans feeding of E. coli strains engineered to produce the double-stranded RNA can trigger RNAi, in the insect Drosophila the feeding of yeast cells engineered to make double-stranded RNA failed to work.

Also, Rajagopal et al. (2002) described that in Spodoptera litura insects, experiments to introduce dsRNA into neonate larvae of S. litura by soaking them in dsRNA solution or by feeding through diet were unsuccessful, since no reduction in transcript levels was detected.

Rao et al. (1998) describe that expression of a snowdrop lectin in transgenic rice plants can confer resistance to rice brown planthopper.

Hence, the prior art does not show that naked, unpackaged dsRNA or siRNA can be used to obtain gene silencing in insects through feeding.

### SUMMARY OF THE INVENTION

The current invention provides means to silence insect genes by using unpackaged dsRNA, in one embodiment such dsRNA is present in vascular tissue, preferably phloem, more particularly phloem sap, and the insect is a plant sap-sucking insect such as an aphid or a whitefly.

The invention described herein is summarized in the following numbered paragraphs:

1. A chimeric gene comprising the following operably linked DNA:

(a) a plant-expressible promoter;

(b) a DNA region which when transcribed yields a double-stranded RNA molecule capable of reducing the expression of an essential gene of a plant sap-sucking insect, said RNA molecule comprising a first and second RNA region wherein:

(i) said first RNA region comprises a nucleotide sequence of at least 19 consecutive nucleotides having at least about 94% sequence identity to the nucleotide sequence of said endogenous gene;

(ii) said second RNA region comprises a nucleotide sequence complementary to said at least 19 consecutive nucleotides of said first RNA region;

(iii) said first and second RNA region are capable of base-pairing to form a double stranded RNA molecule between at least said 19 consecutive nucleotides of said first and second region;

(c) optionally, a 3′ end region comprising transcription termination and polyadenylation signals functioning in cells of said plant.

2. The chimeric gene of paragraph 1, wherein said essential gene of said plant sap-sucking insect is selected from the group consisting of the genes encoding the following: a gut cell protein, a membrane protein, a transcription factor, an ecdyson receptor, a vATPase, an amino acid transporter, a peptidylglycine alpha-amidating monooxygenase; a cystein protease, an aminopeptidase, a dipeptidase, a sucrase/transglucosidase, a translation elongation factor, the eucaryotic translation initiation factor 1A, a splicing factor, an apoptosis inhibitor, a tubulin protein, an actin protein, an alpha-actinin protein, a histone, a histone deacetylase, a cell cycle regulatory protein, a cellular respiratory protein; a receptor for an insect-specific hormonal signal, a juvenile hormone receptor, an insect peptidic hormone receptor; a protein regulating ion balance in the cell, a proton-pump, a Na/K pump, an intestinal protease; an enzyme involved in sucrose metabolism, a digestive enzyme, a trypsin-like protease and a cathepsin B-like protease.

3. The chimeric gene of paragraphs 1 or 2, wherein said double-stranded RNA silences the gene corresponding to the DNA sequence of any one of SEQ ID NO: 5 to 8, SEQ ID NO: 11 or SEQ ID NO:12.

4. The chimeric gene of any one of paragraphs 1 to 3, wherein between said first and second RNA region, a spacer region containing a plant intron is present.

5. The chimeric gene of any one of paragraph 1 to 4, wherein said essential gene has a portion which occurs with the same sequence or with at least 94% sequence identity in homologous genes of several plant sap-sucking insects.

6. The chimeric gene of any one of paragraph 1 to 5, wherein said promoter is a constitutive promoter.

7. The chimeric gene of any one of paragraph 1 to 6, wherein said promoter is a vascular-specific or a phloem-specific promoter.

8. The chimeric gene of paragraph 7, wherein vascular- or phloem-specific promoter is selected from the group consisting of: the rolC or rolA promoter of Agrobacterium rhizogenes, the promoter of the Agrobacterium tumefaciens T-DNA gene 5, the rice sucrose synthase RSs1 gene promoter, the Commelina yellow mottle badnavirus promoter, the coconut foliar decay virus promoter, the rice tungro bacilliform virus promoter, the promoter of the pea glutamine synthase GS3A gene, the invCD111 and invCD141 promoters of the potato invertase genes, the promoter isolated from Arabidopsis shown to have phloem-specific expression in tobacco by Kertbundit et al (1991), the VAHOX1 promoter region, the pea cell wall invertase gene promoter, an acid invertase gene promoter from carrot, the promoter of the sulfate transporter gene Sultr1;3, the promoter of a plant sucrose synthase gene, the promoter of a plant sucrose transporter gene.

9. A plant cell, tissue, or a plant or a plant seed comprising the chimeric gene of any one of paragraphs 1 to 8 or the double-stranded RNA molecule described in any one of paragraphs 1 to 8.

10. A method to silence a gene of a plant sap-sucking insect, comprising applying to the feed of said plant sap-sucking insect unpackaged, naked dsRNA or siRNA which is targeted to an essential plant sap-sucking gene.

11. The method of paragraph 10, wherein said essential gene is any of the genes listed in paragraph 2 above.

12. The method of paragraph 10, wherein said application is by expression of a dsRNA chimeric gene in phloem cells of a plant.

13. A method to silence a gene in a plant sap-sucking insect, comprising: adding naked, unpackaged dsRNA or siRNA to the diet or feed of said plant sap-sucking insect, wherein said dsRNA or siRNA targets said gene.

14. A method of controlling plant sap-sucking insects, comprising expressing in the phloem of a plant dsRNA that targets an essential plant sap-sucking insect gene.

15. The method of paragraph 14 wherein said gene is a gene expressed at least in the intestine or in gut cells.

16. The method of paragraph 14 wherein said plant sap-sucking insect is an aphid or a whitefly.

17. A plant, comprising stably inserted in its genome, the chimeric gene of paragraph 1, so that said chimeric gene is expressed in the phloem or xylem of said plant.

18. A method of identifying gene function in a plant sap-sucking insect, comprising the step of applying naked, unpackaged dsRNA targeting a plant sap-sucking insect gene to the diet of said insect, and evaluating phenotypic or biochemical changes in said insect.

19. A method of identification of novel targets for insecticidal compounds, comprising the steps of: a) applying naked, unpackaged dsRNA or siRNA molecules to the feed or diet of a plant-sap sucking insect; b) analyzing which genes when silenced confer lethality to said insect, c) cloning and characterizing said genes thus analyzed; d) identifying compounds that disrupt or inactivate said gene or the RNA or protein encoded thereby; and e) contacting said compounds with said insect or feed or diet of said insect to confirm the pesticidal nature of said compound.

20. Phloem of a plant, comprising siRNA targeted to an aphid essential gene.

21. Phloem sap of a plant, comprising siRNA targeted to an aphid essential gene.

22. An aphid gene comprising the sequence of any one of SEQ ID NO:5 to 8, SEQ ID NO: 11 or SEQ ID NO:12.

23. The method of paragraph 18 or 19, wherein a cationic oligopeptide is mixed in the diet together with the dsRNA.

24. The method of paragraph 23, wherein said oligopeptide is a 12 amino acids poly-Arginine peptide.

25. The plant cell, tissue, plant or plant seed of paragraph 9 or 17, which also comprises a chimeric gene encoding a cationic oligopeptide.

26. The plant cell, tissue, plant or plant seed of paragraph 25, wherein said oligopeptide is a 12 amino acids poly-Arginine peptide.

### DETAILED DESCRIPTION OF THE INVENTION

In accordance with the invention, dsRNA or siRNA is fed to a plant sap-sucking insect without being contained in a cell or a transfection-promoting agent. In one embodiment of the invention, this feeding is on a plant which expresses the dsRNA or siRNA so that it enters the sap contained in its vascular system. A “transfection promoting agent”, as used herein, refers to a lipid-containing material that allows or enhances passage of the cell membrane and hence secures uptake by a cell of an extracellular molecule or compound such as a dsRNA, particularly liposomes. Such agents are described in published PCT patent application WO 03/004644. For the avoidance of doubt, the cationic oligopeptides used in some embodiments of the current invention are not included in this definition of transfection-promoting agents. A dsRNA or siRNA delivered without transfection promoting agent is also referred to herein as “naked” and/or “unpackaged” dsRNA or siRNA. A cationic oligopeptide, as used herein, does not “package” dsRNA or siRNA (contrary to a transfection promoting agent, such as a liposome) and hence can be supplied together with the dsRNA of siRNA in the case of “unpackaged” delivery of dsRNA or siRNA. The term “chimeric” when referring to a gene or DNA sequence is used to refer to a gene or DNA sequence comprising at least two functionally relevant DNA fragments (such as promoter, 5′UTR, coding region, 3′UTR, intron) that are not naturally associated with each other and/or originate, for example, from different sources. “Foreign” referring to a gene or DNA sequence with respect to a plant species is used to indicate that the gene or DNA sequence is not naturally found in that plant, or is not naturally found in that genetic locus in that plant. The term “foreign DNA” will be used herein to refer to a DNA sequence as it has incorporated into the genome of a plant as a result of transformation.

Two sequences or genes (or parts thereof) which are “homologous” or “similar”, as used herein, are similar in sequence to such a degree that when the two sequences are aligned, the percent sequence identity, i.e., the number of positions with identical nucleotides divided by the number of nucleotides in the shorter of the sequences, is higher than 70%, higher than 85%, higher than 90%, higher than 95%, or is between 96% and 100%. Homologous genes or parts thereof, as used herein, do not require any evolutionary relationship, though genes related to each other by independent evolution from the same ancestral gene can be homologous genes as used herein. As used herein, a gene “homolog” (or homologous gene) can be a gene paralog (or a paralogous gene) and a gene ortholog (or an orthologous gene). In one embodiment of the invention a homologous gene is an orthologous gene (i.e., a similar gene in a different species likely evolved from a common ancestor and which normally has retained essentially the same or the same function). Sequences or parts of sequences which have “high sequence identity”, as used herein, refers to the number of positions with identical nucleotides divided by the number of nucleotides in the shorter of the sequences, being higher than higher than 95%, or between 96% and 100%. A target gene, or at least a part thereof, as used herein, preferably has high sequence identity to the dsRNA of the invention in order for efficient gene silencing to take place in the target pest. Identity in sequence of the dsRNA or siRNA with a part of the target gene RNA is included in the current invention but is not necessary.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch 1970). A computer-assisted sequence alignment can be conveniently performed using a standard software program such as GAP which is part of the Wisconsin Package Version 10.1 (Genetics Computer Group, Madison, Wis., USA) using the default scoring matrix with a gap creation penalty of 50 and a gap extension penalty of 3.

For the purpose of the invention, the “complement of a nucleotide sequence X” is the nucleotide sequence which would be capable of forming a double stranded DNA molecule with the represented nucleotide sequence, and which can be derived from the represented nucleotide sequence by replacing the nucleotides by their complementary nucleotide according to Chargaff's rules (A< >T; G< >C) and reading in the 5′ to 3′ direction, i.e., in opposite direction of the represented nucleotide sequence.

As used herein, “dsRNA” refers to double-stranded RNA that comprises a sense and an antisense portion of a selected target gene (or sequences with high sequence identity thereto so that gene silencing can occur), as well as any smaller double-stranded RNAs formed therefrom by RNAse or dicer activity. Such dsRNA can include portions of single-stranded RNA, but contains at least 19 nucleotides double-stranded RNA. In one embodiment of the invention, the dsRNA is a hairpin RNA which contains a loop or spacer sequence between the sense and antisense sequences of the gene targeted, preferably such hairpin RNA spacer region contains an intron, particularly the rolA gene intron (Pandolfini et al., 2003), the dual orientation introns from pHellsgate 11 or 12 (see WO 02/059294 (incorporated by reference herein), and SEQ ID NO: 25 and 15 therein) or the pdk intron (Flayeria trinervia pyruvate orthophosphate dikinase intron 2; see WO99/53050 incorporated by reference). “siRNAs” as used herein are small interfering (double-stranded) RNA molecules of 16-30 bp, 19-28 bp, or 21-26 bp, e.g., the RNA forms that can be created by RNAseIII or dicer activity from longer dsRNA. “siRNAs” as used herein include any double-stranded RNA of 19 to 26, or 21 to 24 basepairs that can interfere with gene expression when present in a cell wherein such gene is expressed. siRNA can be synthetically made, expressed and secreted directly from a transformed cell or can be generated from a longer dsRNA by enzymatic activity. These siRNAs can be blunt-ended or can have overlapping ends.

In one embodiment of the invention, sense and antisense RNAs can be separately expressed in vitro or in host cells, e.g., in cells of a plant from different chimeric gene constructs using the same or a different promoter or from a construct containing two convergent promoters in opposite orientation. These sense and antisense RNAs which are formed, e.g., in the same plant cells, can then combine to form dsRNA or siRNA. It is clear that whenever reference is made herein to a dsRNA or siRNA chimeric gene or a dsRNA or siRNA molecule, that such dsRNA or siRNA formed, e.g., in plant cells, from sense and antisense RNA produced separately is also included. Also synthetically made siRNA or dsRNA annealing RNA strands are included herein when the sense and antisense strands are present together.

A dsRNA or siRNA “targeting” a plant sap-sucking insect gene, as used herein, refers to a dsRNA or siRNA that is designed to be identical or have high sequence identity to an endogenous plant sap-sucking insect gene (a target gene), and as such is designed to silence such gene upon application to such insect. One dsRNA can target one or several homologous genes in one plant sap-sucking insect or one or several homologous genes in different plant sap-sucking insects which can feed on the same host plant.

The dsRNA chimeric gene, encoding a dsRNA targeting a plant sap-sucking essential gene, can be stably inserted in a conventional manner into the genome of a single plant cell, and the so-transformed plant cell can be used in a conventional manner to produce a transformed plant that has increased insect resistance. In this regard, a disarmed Ti-plasmid, containing the dsRNA chimeric gene, in Agrobacterium tumefaciens can be used to transform the plant cell, and thereafter, a transformed plant can be regenerated from the transformed plant cell using the procedures described in the art, for example, in EP 0 116 718, EP 0 270 822, PCT publication WO 84/02913 and published European Patent application (“EP”) 0 242 246. Preferred Ti-plasmid vectors each contain the dsRNA chimeric gene between the border sequences, or at least located to the left of the right border sequence, of the T-DNA of the Ti-plasmid. Of course, other types of vectors can be used to transform the plant cell, using procedures such as direct gene transfer (as described, for example in EP 0 233 247), pollen mediated transformation (as described, for example in EP 0 270 356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., 1990; Gordon-Kamm et al., 1990) and rice (Shimamoto et al., 1989; Datta et al., 1990) and the method for transforming monocots generally (PCT publication WO 92/09696). For cotton transformation, especially preferred is the method described in PCT patent publication WO 00/71733. For soybean transformation, reference is made to methods known in the art, e.g., Hinchee et al. (1988) and Christou et al. (1990) or the method of WO 00/42207.

The resulting transformed plant can be used in a conventional plant breeding scheme to produce more transformed plants with the same characteristics or to introduce the dsRNA gene in other varieties of the same or related plant species. Seeds, which are obtained from the transformed plants, contain the dsRNA gene as a stable genomic insert. Plants comprising a dsRNA or siRNA in accordance with the invention include plants comprising or derived from root stocks of plants comprising the dsRNA chimeric gene of the invention, e.g., fruit trees. Hence, any non-transgenic grafted plant parts inserted on a transformed plant or plant part are included in the invention since the RNA interference signal is transported to these grafted parts and any aphids feeding on such grafted plant are similarly affected by the dsRNA or siRNA of the invention.

A DNA encoding a dsRNA is inserted in a plant cell genome so that this DNA is downstream (i.e., 3′) of, and operably linked to, a plant-expressible promoter which can direct expression in plant cells. This is preferably accomplished by inserting the dsRNA chimeric gene in the plant cell genome, particularly in the nuclear or plastid (e.g., chloroplast) genome.

A ‘plant-expressible promoter’ as used herein refers to a promoter that ensures expression of a dsRNA of the invention in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., 1981), CabbB-S (Franck et al., 1980) and CabbB-JI (Hull and Howell, 1987); promoters from the ubiquitin family (e.g., the maize ubiquitin promoter of Christensen et al., 1992, see also Comejo et al., 1993), the gos2 promoter (de Pater et al., 1992), the emu promoter (Last et al., 1990), actin promoters such as the promoter described by An et al. (1996), the rice actin promoter described by Zhang et al. (1991); promoters of the Cassaya vein mosaic virus (NO 97/48819, Verdaguer et al. (1998)), the pPLEX series of promoters from Subterranean Clover Stunt Virus (WO 96/06932, particularly the S4 or S7 promoter), a alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T-DNA (Velten et al., 1984). Alternatively, a plant-expressible promoter can be a tissue-specific promoter, i.e., a promoter directing a higher level of expression in some cells or tissues of the plant, e.g., in green tissues (such as the promoter of the PEP carboxylase). The plant PEP carboxylase promoter (Pathirana et al., 1997) has been described to be a strong promoter for expression in vascular tissue and is useful in one embodiment of the current invention. Alternatively, a plant-expressible promoter can also be a wound-inducible promoter, such as the promoter of the pea cell wall invertase gene (Zhang et al., 1996). A ‘wound-inducible’ promoter as used herein means that upon wounding of the plant, either mechanically or by insect feeding, typically by piercing of the plant to access the vascular system in the plant sap-sucking insects, expression of the coding sequence under control of the promoter is significantly increased in such plant. It has been shown that plant sap-sucking insects can also cause plant defense responses similar to those observed for other pathogens and wounding (Moran and Thompson, 2001). Promoters of certain of such genes induced by plant sap-sucking insects, preferably when their expression is preferentially in the vascular tissue, particularly phloem, can also be used in accordance with the current invention.

In one embodiment of this invention the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells. Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the promoters selected from the group consisting of: the SCSV3, SCSV4, SCSV5, and SCSV7 promoters (Schunmann et al., 2003), the rolC gene promoter of Agrobacterium rhizogenes (Kiyokawa et al., 1994; Pandolfini et al., 2003; Graham et al., 1997; Guivarc'h et al., 1996, Almon et al; 1997), the rolA gene promoter of Agrobacterium rhizogenes (Dehio et al., 1993), the promoter of the Agrobacterium tumefaciens T-DNA gene 5 (Korber et al. 1991), the rice sucrose synthase RSs1 gene promoter (Shi et al., 1994), the CoYMV or Commelina yellow mottle badnavirus promoter (Medberry et al., 1992; Zhou et al., 1998), the CFDV or coconut foliar decay virus promoter (Rohde et al., 1994; Hehn and Rhode, 1998), the RTBV or rice tungro bacilliform virus promoter (Yin and Beachy, 1995; Yin et al., 1997), the pea glutamine synthase GS3A gene (Edwards et al., 1990; Brears et al., 1991), the invCD111 and invCD141 promoters of the potato invertase genes (Hedley et al., 2000), the promoter isolated from Arabidopsis shown to have phloem-specific expression in tobacco by Kertbundit et al (1991), the VAHOX1 promoter region (Tomero et al., 1996), the pea cell wall invertase gene promoter (Zhang et al., 1996), the promoter of the endogenous cotton protein related to chitinase of US published patent application 20030106097, an acid invertase gene promoter from carrot (Ramloch-Lorenz et al., 1993), the promoter of the sulfate transporter gene Sultr1;3 (Yoshimoto et al., 2003), a promoter of a sucrose synthase gene (Nolte and Koch, 1993), and the promoter of a tobacco sucrose transporter gene (Kuhn et al., 1997). Also, any promoter homologous to (or having a high sequence identity to) any of the above promoters and also exhibiting phloem-specific expression, as defined herein, can be used. The selection of a particular promoter can depend on the insect species mainly targeted (e.g., a specific insect targeted can be mostly a leaf feeder or mostly a root feeder, allowing different promoter specificities to be used), and on the expression level and tissue distribution desired. These promoters can be combined with enhancer elements, they can be combined with minimal promoter elements, or can comprise repeated elements to ensure the expression profile desired.

Elements which can be used to increase expression in plant cells can be: introns at the 5′ end or 3′ end of the chimeric gene, e.g., the hsp70 intron, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

The dsRNA gene of the invention can be inserted in the plant genome so that the inserted gene part is upstream (i.e., 5′) of suitable 3′ end transcription regulation signals (i.e., transcript formation and polyadenylation signals). This is preferably accomplished by inserting the dsRNA chimeric gene in the plant cell genome. Preferred polyadenylation and transcript formation signals include those of the nopaline synthase gene (Depicker et al., 1982), the octopine synthase gene (Gielen et al., 1984), the SCSV or the Malic enzyme terminators (Schunmann et al., 2003), and the T-DNA gene 7 (Velten and Schell, 1985), which act as 3′-untranslated DNA sequences in transformed plant cells.

The dsRNA chimeric gene can optionally be inserted in the plant genome as a hybrid gene, containing several dsRNA regions which target different genes in the same or different plant sap-sucking insects, or which target different portions of the same gene. Also, it is convenient to include in the transforming DNA of the invention also a selectable or scorable marker gene, such as the bar or the neo gene, so that transformed plants can easily be selected by application of glufosinate or kanamycin, respectively, as is well known in the art.

Although plant delivery of a dsRNA or siRNA is an embodiment of this invention, in accordance with this invention, application of the dsRNA or siRNA of the invention to a plant sap-sucking insect can be done in several ways, and need not be by way of a plant expressing a dsRNA or siRNA. Any method of delivery of siRNA or dsRNA not contained in a cell or a cell-transfection agent (such as a liposome) is included herein, e.g., in vitro produced siRNA or dsRNA applied to an insect diet or feed.

“Insecticidal activity” of a dsRNA or siRNA, as used herein, refers to the capacity to obtain mortality in insects when such RNA is fed to insects, preferably by expression in a recombinant host such as a plant, which mortality is significantly higher than the controls (using a non-insect dsRNA or buffer). “Insect-control” of a dsRNA or siRNA, as used herein, refers to an amount of RNA which is sufficient to limit damage on a plant by insects feeding on such plant, e.g., by killing the insects or by inhibiting the insect development, fertility or growth in such a manner that they provide less damage to a plant, produce fewer offspring, are less fit or more susceptible to predator attack, or that insects are even deterred from feeding on such plant.

Information on how to design optimal dsRNA or siRNA sequences once a target gene is known can be found with commercial providers, e.g., the companies Ambion and Cenix BioScience (Ambion Inc., 2130 Woodward Street, Austin, Tex. 78744-1832, USA; and see www.ambion.com; and Cenix BioScience GmbH, Pfotenhauerstr. 108, 01307 Dresden, Germany, see www.cenix-bioscience.com). Preferably, the dsRNAs or siRNAs to be used in this invention target at least one essential plant sap-sucking insect gene, or an essential plant sap-sucking insect gene occurring without significant sequence divergence (at least in a certain region) in a range of plant sap-sucking insect pests of the host plant concerned. In one embodiment of this invention, such dsRNAs or siRNAs do not silence genes of the plant host or of other non-target animals, such as plant sap-sucking insect predators (e.g., ladybird larvae, anthocorid bugs, lacewing, parasitic wasps or hoverfly larvae) or animals such as reptiles, amphibians, birds, or mammals. This can be analyzed in available databases, e.g., by a BLAST search (see also www.ncbi.nim.nih.gov/BLAST) or by hybridization with existing DNA libraries of representative non-target organisms. In this respect, when using the A. gossypii eIF1A sequence of SEQ ID NO: 5 to transform a plant with a dsRNA chimeric gene, preferably only that portion from nucleotide position 72 to the end in SEQ ID NO:5 should be used as gene target in designing the dsRNA molecule, or at least the portion from nucleotide position 50 to 73 in SEQ ID NO:5 should be avoided in the dsRNA. In one embodiment, a portion of a target sequence is selected which is present in several plant sap-sucking insects of a plant host in identical sequence or with high sequence identity, e.g., a part of a class of gene(s) with high sequence homology in several plant sap-sucking insect pests.

A plant sap-sucking insect “target gene”, “an essential gene”, or “a gene essential for a plant sap-sucking insect”, as used herein, is a gene the silencing of which brings about a decreased growth, development, reproduction or survival of a plant sap-sucking insect. In one embodiment, the partial or complete silencing of an essential gene of a plant sap-sucking insect results in significant insect mortality or significant insect control when such gene is silenced by dsRNA or siRNA compared to control insects fed on dsRNA or siRNA targeting a non-essential gene or a gene not expressed in the plant sap-sucking insect. In one embodiment of this invention, the dsRNA or siRNA of the invention corresponds to an exon in the target gene.

In one embodiment of the invention genes expressed in cells of the plant sap-sucking insect gut tissue or in the midgut are targeted, preferably genes involved in gut cell metabolism, growth or differentiation. These genes can encode plant sap-sucking insect gut membrane proteins such as transporter molecules or ion pumps, e.g., a vATPase (e.g., a homolog to the Drosophila gene of Genbank accession number AF143200) or an amino acid transporter gene. Useful plant sap-sucking insect target genes in accordance with the invention also include genes encoding the following: a transcription factor; a peptidylglycine alpha-amidating monooxygenase (e.g., a homolog of the Drosophila phm gene described in Jiang et al., 2000); a cystein protease (Cristofoletti et al., 2003), an aminopeptidase (e.g., a gene homologous to a aminopeptidase N gene expressed in the gut of Drosophila, or a gene encoding the pea aphid aminopeptidase N (Rahbé et al., 1995; Cristofoletti et al., 2003), a dipeptidase, a sucrase/transglucosidase (see, Ashford et al. 2000; Cristofoletti et al. (2003)); a translation initiation factor (such as the eucaryotic translation initiation factor 1A (eIF1A) (e.g., based on the homolog in Drosophila (Myrick and Dearolf, 2000, Genbank accession number AF169359)), a translation elongation factor (such as a plant sap-sucking insect gene homologous to the Drosophila elongation factor 1 alpha gene (Hovemann et al., 1988, Genbank accession number X06869)); a splicing factor (such as a gene homolog of the Drosophila SF1 gene (Marzoui et al., 1999), or Genbank accession number NM_{—}079915); an IAP inhibiting apoptosis (e.g., a homolog of the IAP gene described in Hay, 2000 or Genbank accession number AA801628); an (alpha) tubulin (e.g., the homolog of the Drosophila alpha-tubulin gene decribed in Matthews and Kaufman, 1987 or in Genbank accession number All 24284), an actin or alpha-actinin (e.g., the homolog to the Drosophila gene described in Fyrberg et al., 1998; Dubreuil and Wang, 2000, and in Genbank accession number NM_{—}058137); a histone protein (e.g., a homolog of the H2A.F/Z family of Drosophila histone genes (Clarkson and Saint, 1999), such as H2AvD (van Daal and Elgin, 1992, Clarkson et al., 1999; Genbank accession number NM_{—}079795); a histone deacetylase (such as a homolog of the histone deacetylases HDAC1 (Mottus et al., 2000); HDAC3 (Johnson et al, 1998) or HDAC4a (Zeremsky et al., 2003), Genbank accession number AF538713); a cell cycle protein; a protein essential for cellular respiration; a receptor for an insect-specific hormonal signal, a juvenile hormone receptor (e.g., a plant sap-sucking juvenile hormone receptor gene described in published PCT patent applications WO99/36520 and WO01/02436), an insect peptidic hormone receptor; plant sap-sucking insect homologous genes of the dmHelicase, dmPITP or dmSPL genes of Drosophila identified in WO01/42479; a (part of) ecdysone receptor (e.g., a plant sap-sucking ecdysone receptor gene as the M. persicae or B. tabaci genes described in published PCT patent applications WO99/36520 and WO01/02436; or an insect gene homologous to the Drosophila ecdyson receptor gene (see, e.g., the ecdyson receptor gene forms described in Bender et al. (1997); Lam and Thummel, 2000), or to the Drosophila ultraspiracle gene (Henrich et al., 2000)); a protein essential for regulating ion balance in the cells (e.g., a proton-pump, a NA/K pump, etc.); an intestinal protease; etc. Possible target genes are also other genes encoding enzymes involved in sucrose metabolism in the plant sap-sucking insect, preferably in the gut, or genes encoding digestive enzymes such as the trypsin-like protease and the cathepsin B-like protease that were recently described in a Homopteran plant sap-sucking insect, the rice brown planthopper (Foissac et al., 2002), and homologous genes found in other plant sap-sucking insects.

Preferred target sequences in accordance with this invention are the plant sap-sucking insect genes identified using the primers of any one of SEQ ID NO:1-4 and SEQ ID NO:9 and 10, or plant sap-sucking insect genes corresponding to or comprising any one of the sequences of SEQ ID NO:5 to 8, SEQ ID NO:11 or SEQ ID NO:12, as well as aphid genes homologous to or having high sequence identity with the sequences of SEQ ID NO:5 to 8, SEQ ID NO:11 or SEQ ID NO:12, this includes other portions of the same aphid genes or genes of other plant sap-sucking insects having high sequence identity or homology to the sequences of SEQ ID NO:5 to 8, SEQ ID NO:11 or SEQ ID NO:12. It is preferred that only portions of the target gene which are known, i.e., those portions of SEQ ID NO:5 to 8, SEQ ID NO:11 or SEQ ID NO:12 that have no “n” positions, are used in the design of the chimeric genes of the invention.

In one embodiment of this invention, target genes are plant sap-sucking insect genes homologous to a gene which when partially or completely silenced (or otherwise prevented to express a functional protein or RNA) in an insect, e.g., Drosophila, results in a mutant with a lethal phenotype (see, e.g., www.fruitfly.org/p_disrupt/; Spradling et al., 1999; and Adams and Sekelsky, 2002 and references cited therein), particularly when such gene is expressed in insect gut cells, particularly in the gut cells lining the gut lumen, especially gut cells lining the midgut. Homologs of Drosophila genes are easily found by existing techniques, e.g., PCR amplification of the plant sap-sucking insect homologous gene using primers targeting a Drosophila essential gene (e.g., in cDNA or genomic libraries of a preferred target insect), or by routine similarity searches in available DNA sequence databases of plant sap-sucking insects.

Target genes can also be found using similar sequences of essential insect genes isolated and characterized in other non-Drosophila insects. In one embodiment of the invention, the target gene preferably produces a stable mRNA in the aphid. Also, in one embodiment of the invention, although the target gene has to be transcribed in the target insect, for obtaining an optimal silencing effect, it is preferred that the target gene does not produce abundant amounts of RNA.

To test performance of a certain dsRNA or siRNA in plants, the system as described in published PCT application WO 03/052108 can be used, wherein plants produce dsRNA/siRNA and effects on the aphids growing on such plants can be assessed. As a control, a different non-essential gene that is absent in aphids, such as a gene encoding a gfp (green fluorescent protein)-specific dsRNA, is tested in parallel in the same viral construct.

Also, in the dsRNA chimeric gene of the invention a nuclear localization signal can be added as described in published US patent application 20030180945 (incorporated herein by reference).

As used herein, nucleotide sequences of RNA molecules may be identified by reference to DNA nucleotide sequences of the sequence listing. However, the person skilled in the art will understand whether RNA or DNA is meant depending on the context. Furthermore, the nucleotide sequence is identical except that the T-base is replaced by uracil (U) in RNA molecules.

The length of the first (e.g., sense) and second (e.g., antisense) nucleotide sequences of the dsRNA molecules of the invention may vary from about 10 nucleotides (nt) up to a length equaling the length in nucleotides of the transcript of the target gene. The length of the first or second nucleotide sequence of the dsRNA of the invention can be at least 15 nt, or at least about 20 nt, or at least about 50 nt, or at least about 100 nt, or at least about 150 nt, or at least about 200 nt, or at least about 500 nt, or at least about 1600 nt. If not all nucleotides in a target gene sequence are known, it is preferred to use such portion for which the sequence is known and which meets other beneficial requirements of the invention.

It will be appreciated that the longer the total length of the first nucleotide sequence in the dsRNA of the invention is, the less stringent the requirements for sequence identity between the total sense nucleotide sequence and the corresponding sequence in the target gene becomes. The total first nucleotide sequence can have a sequence identity of at least about 75% with the corresponding target sequence, but higher sequence identity can also be used such as at least about 80%, at least about 85%, at least about 90%, at least about 95%, about 100%. The first nucleotide sequence can also be identical to the corresponding part of the target gene. However, it is advised that the first nucleotide sequence includes a sequence of 19 or 20, or about 19 or about 20 consecutive nucleotides, or even of about 50 consecutive nucleotides, or about consecutive 100 nucleotides, or about 150 consecutive nucleotides with only one mismatch, preferably with 100% sequence identity, to the corresponding part of the target gene. For calculating the sequence identity and designing the corresponding first nucleotide sequence, the number of gaps should be minimized, particularly for the shorter sense sequences.

The length of the second (e.g., antisense) nucleotide sequence in the dsRNA of the invention is largely determined by the length of the first (e.g., sense) nucleotide sequence, and may correspond to the length of the latter sequence. However, it is possible to use an antisense sequence which differs in length by about 10% without any difficulties. Similarly, the nucleotide sequence of the antisense region is largely determined by the nucleotide sequence of the sense region, and may be identical to the complement of the nucleotide sequence of the sense region. Particularly with longer antisense regions, it is however possible to use antisense sequences with lower sequence identity to the complement of the sense nucleotide sequence, such as at least about 75% sequence identity, or least about 80%, or at least about 85%, more particularly with at least about 90% sequence identity, or at least about 95% sequence to the complement of the sense nucleotide sequence. Nevertheless, it is advised that the antisense nucleotide sequence always includes a sequence of 19 or 20, about 19 or about 20 consecutive nucleotides, although longer stretches of consecutive nucleotides such as about 50 nucleotide, or about 100 nucleotides, or about 150 nucleotides with no more than one mismatch, preferably with 100% sequence identity, to the complement of a corresponding part of the sense nucleotide sequence can also be used. Again, the number of gaps should be minimized, particularly for the shorter (19 to 50 nucleotides) antisense sequences.

In one embodiment of the invention, the DNA molecules according to the invention may comprise a DNA region encoding a spacer between the DNA region encoding the first and second nucleotide sequences. As indicated in WO 99/53050 the spacer may contain an intron to enhance gene silencing. A particularly preferred intron functional in cells of plants is the pdk intron (Flayeria trinervia pyruvate orthophosphate dikinase intron 2; see WO99/53050 incorporated by reference), the delta 12 desaturase intron from Arabidopsis (Smith et al., 2000) or the intron of the rolA gene (Magrelli et al., 1994; Spena and Langenkemper, 1997).

In one embodiment of the invention, the dsRNA molecule may further comprise one or more regions having at least 94% sequence identity to regions of at least 19 consecutive nucleotides from the sense nucleotide sequence of the target gene, different from the at least 19 consecutive nucleotides as defined in the first region, and one or more regions having at least 94% sequence identity to at least 19 consecutive nucleotides from the complement of the sense nucleotide sequence of the target gene, different from the at least 19 consecutive nucleotides as defined in the second region, wherein these additional regions can basepair amongst themselves.

Plants to which the current invention can be applied include but are not limited to the following plants: corn, cotton, rice, soybean, Brassica species plants, Brassica napus, cauliflower, carrot, pea, wheat, barley, rye, tomato, potato, sugarbeet, cut flowers, roses, fruit plants (apple, pear, peach, strawberry, etc.), trees (such as poplar and willow), and lettuce. In one embodiment of the invention, the plants to which the invention is applied are cotton or rice plants. When cotton is the host plant of the invention the dsRNA preferably targets genes in one or all of the following sucking insects: Aphis gossypii, Myzus persicae, Lygus bugs, whitefly, stink bugs, thrips, and Creontiades dilutus, particularly Aphis gossypii, Myzus persicae and Creontiades dilutus.

Together with the strategy of this invention, it is preferred to use other tactics for aphid control, such as expression of a snowdrop (Galanthus nivalis) lectin as described by Down et al. (1996), Stoger et al. (1999) and Rao et al. (1998) or the mannose-binding lectins of Roy et al. (2002) in the plants of the invention, preferably in the phloem of the plants of the invention, and/or the expression of a protease inhibitor such as the soybean Kunitz trypsin inhibitor (Foissac et al., 2002), or the variant mustard trypsin inhibitor Chy8 (Ceci et al., 2003) and similar protease inhibitors active against plant sap-sucking insects, particularly aphids. Also the timely application of effective chemical or biological insecticides, and the use of natural aphid predators are possible tactics to use together with the plants of the invention. Also existing endogenous resistance genes such as the tomato Mi nematode-resistance gene (which also confers resistance to certain aphid species, see Rossi et al., 1998) or the VAT gene (WO 2004072109) can be used to protect a plant of the invention against aphids and provide different mechanisms of resistance, hence minimizing chances of insect resistance development.

Also, in one embodiment of this invention, the plant co-expresses antibacterial proteins or peptides, preferably in the phloem, to kill or negatively affect symbiontic bacteria occurring in aphids that are believed to provide the aphids with certain essential compounds (such as amino acids) which they may not get sufficiently from feeding on plant sap. These proteins can be any one of the antibacterial peptides or proteins known in the art which are effective in inhibiting bacterial growth, and which are preferably provided with a signal for extracellular or for phloem targeting. Such a protein signal sequence can be found in many proteins which are targeted to the phloem, e.g., the GNA snowdrop lectin described above. Also, a dsRNA can be expressed in the plants of the invention, which dsRNA targets an essential gene of such symbiontic bacteria, e.g., the essential genes described by Shigenobu et al. (2000) for the genome of the bacterial symbiont (Buchnera sp.) of the pea aphid, and homologous forms for symbionts in other aphids.

In one embodiment of this invention, together with the dsRNA or siRNAs of the current invention, a chimeric gene encoding a cationic oligopeptide is expressed in the plants of the invention. With a “cationic oligopeptide” as used herein, is meant an oligopeptide of larger then 5 and smaller then 40 amino acids with a net positive charge, and the ability to facilitate transport of siRNA or dsRNA across an insect cell membrane. Such cationic oligopeptides are preferably between 5 and 40 amino acids long, between 10 and 30 amino acids long, particularly of about 12-18 amino acids long, more particularly 12-18 amino acids long, and can bind to the dsRNA of the invention and besides stabilizing the dsRNA, they facilitate the entry of the dsRNA into the sap-sucking insect cells, particularly their gut cells, preferably their midgut cells. In this embodiment, such a cationic oligopeptide, a sequence of multiple such cationic peptides separated by spacer amino acids which are cleavable inside or outside the cell, or a cationic oligopeptide fused to a phloem-targeting signal or to another protein (such as the prosystemin fusion described by Tortiglione et al. (2003)) is expressed in the same cells as the dsRNA or siRNA of the invention, preferably but not necessarily using the same promoter (in different chimeric genes), leading to accumulation of the peptide and the dsRNA of the invention in the plant cells and/or phloem. In one embodiment of this invention, the cationic oligopeptide is a poly-Arginine 12-mer (Unnamalai et al., 2004). Other cationic oligopeptides that can be co-expressed in any plant include from 5 to 40, 10 to 30, or 12-18 amino acids long poly-Arginine, poly-Lysine or poly-Histidine peptides or mixtures of any of these 3 basic amino acids, or the penetratin peptide, transportan peptide, TAT peptide, MAP peptide, R7 peptide, pVEC peptide, MPG-delta-NLS peptide, KALA peptide or Buforin 2 peptide, as described in Jarver and Langel (2004, this publication and the cited reference papers per peptide are incorporated herein by reference), or other oligopeptides smaller then 40 amino acids with a net positive charge, and the ability to facilitate transport of siRNA or dsRNA across an insect cell membrane, preferably the MPG-delta-NLS peptide, the 12-amino acids poly-Arginine peptide or the TAT peptide. In one embodiment of this invention, the chimeric gene encoding the dsRNA and the chimeric gene encoding the cationic oligopeptide of the invention are assembled in one transforming DNA, e.g., in one T-DNA insert in an Agrobacterium plasmid, to secure expression from one locus in the plant. In one embodiment of this invention, these cationic oligopeptides are useful for facilitating transfer of any dsRNA or siRNA into the cells of any insect species, not just the sap-sucking insects. Such insects include insects used as model species, or insects pests of corn, cotton, rice, soybean, Brassica species plants, oilseed rape, cabbage, cauliflower, carrot, pea, wheat, barley, rye, tomato, potato, sugarbeet, cut flowers, roses, fruit plants (apple, pear, peach, strawberry, etc.), trees (such as poplar and willow), and lettuce, particularly the european corn borer, cotton bollworms, and corn rootworms, besides the plant sap-sucking insect pests described herein. Particularly such insects are selected from the list consisting of: Drosophila melanogaster, Anopheles spp. insects, Helicoverpa zea, Helicoverpa annigera, Helicoverpa punctigera, Heliothis virescens, Ostrinia nubilalis, Spodoptera frugiperda, Agrotis ipsilon, Pectinophora gossypiella, Scirphophaga incertulas, Cnaphalocrocis medinalis, Sesamia inferens, Chilo partellus, Anticarsia gemmatalis, Plathypena scabra, Pseudoplusia includens, Spodoptera exigua, Spodoptera omithogalli, Epinotia aporema, Rachiplusia nu, Chilo suppressalis, Scirpophaga incertulas, Sesamia inferens, Cnaphalocrocis medinalis, Hereitogramma licarisalis, Naranga aenescens, Mycalesis gotama, Marasmia patnalis, Marasmia exigua, Marasmia ruralis, Nymphula depunctalis, Scirpophaga innotata, Spodoptera litura, Chilo polychrysus, Rupela albinella, Diatraea saccharalis, Spodoptera frugiperda, Mythimna unipuncta, Chilo zacconius and Pamara guffata, most particularly Chilo suppressalis, Scirpophaga incertulas, Marasmia patnalis, Cnaphalocrocis medinalis, Agelastica alni, Hypera postica, Hypera brunneipennis, Haltica tombacina, Anthonomus grandis, Tenebrio moliftor, Triboleum castaneum, Dicladispa armigera, Trichispa serica, Oulema oryzae, Colaspis brunnea, Lissorhorptrus oryzophilus, Phyllotreta cruciferae, Phyllotreta striolata, Psylliodes punctulata, Entomoscelis americana, Meligethes aeneus, Ceutorynchus sp., Psylliodes chrysocephala, Phyllotreta undulata, Leptinotarsa decemlineata, Diabrotica undecimpunctata undecimpunctata, Diabrotica undecimpunctata howardi, Diabrotica barber, and Diabrotica virgifera. Such cationic oligopeptides can also be delivered together with any dsRNA or siRNA molecules to insect cells in vitro or in cell culture (e.g., by adding them to the culture medium), or they may be mixed with dsRNA or siRNA molecules in the insect feed to be fed to insects, with the aim to silence certain insect target genes. Hence, these peptides are a useful research tool for the analysis of gene function, by mixing them (preferably in 1:1 ratio) with dsRNA or siRNA molecules targeting certain insect genes, e.g., in a process of pesticide target development as described above.

In this embodiment, the dsRNA or siRNA then targets an essential gene of this insect, preferably an essential gene that is expressed in the gut, and this dsRNA or siRNA and the cationic oligopeptide of the invention can be administered to an insect species cell culture in vitro, to a live insect, preferably to a larval stage thereof, by spraying in a field or by co-expression in a transgenic plant on which such insect species tends to feed.

In one embodiment of this invention, the current strategy is used in a plant which has already acquired partial or enhanced resistance to plant sap-sucking insects such as aphids or whiteflies, which plant is obtained by normal breeding and selection, or in a hybrid plant having better overall growth and vigour.

The dsRNAs or siRNAs of the current invention, and the method of applying them to the feed of insects such as plant sap-sucking insects, also have applications in insecticide development. New insecticides can be developed using the process of identifying and validating biological targets against which potential ligands can be screened (e.g., Margolis and Duyk, 1998). Production of new insecticides can be done using target-based discovery approaches. Genes that, when partially or entirely inactivated, kill the organism or significantly affect normal development when knocked out or repressed represent interesting targets. To identify compounds that have the same effect on the organism, high-throughput screening assays can be established to test compounds for their ability to interfere in vitro with the normal activity of the target. As such, the invention provides a method of identification of novel targets for insecticidal compounds, comprising the steps of: a) applying naked, unpackaged dsRNA or siRNA molecules to the feed or diet of a plant-sap sucking insect; b) analyzing which genes when silenced confer lethality to said insect, c) cloning and characterizing said genes thus analyzed; and optionally: d) identifying compounds that disrupt or inactivate said gene or the protein encoded thereby; and e) contacting said chemicals with said insect or feed or diet of said insect to confirm the pesticidal nature of said compound. In one embodiment of this invention, this unpackaged dsRNA or siRNA can be supplied together with a cationic oligopeptide as described herein for applications to any insect species.

Preferred target pests are plant sap-sucking insects, particularly of the order Hemiptera, preferably insects of the suborder Homoptera or Heteroptera, preferably aphids and whiteflies, particularly insects of the family of Aphididae. The invention is similarly applicable to any plant sap-sucking insects feeding from xylem by using a constitutive promoter or a xylem-specific promoter. Examples of xylem-specific promoters (i.e., promoter preferentially but not necessarily exclusively active in xylem) include but are not limited to: the Pal2 promoter, e.g. from bean (Leyva et al., 1992), the poxN or poxA promoters from rice (Ito et al., 2000), the PtCesA promoter (Wu et al., 2000), the XCP1 promoter (Funk et al., 2002), the cotton curl leaf virus promoter (Yingqiu et al., 2000), the vs-1 promoter element (Torres-Schumann et al., 1996).

“Plant sap-sucking insects” as used herein are insects feeding on plants using their sharp mouth parts which can be inserted into a plant to take fluid from the plant vascular system, in one embodiment these are insects feeding directly on the fluids in the plant vascular system, preferably insects only feeding on the fluids in the plant vascular system. In the insertion step, plant cells can also be damaged which may or may not be used as a food source by the plant sap-sucking insect. These insects are plant pests because their feeding reduces the vitality of the crops they feed on and they can transmit viral diseases. Also, many such sap-sucking insects secrete a sugar-rich fluid named honeydew that accumulates on lower plant parts, and such parts soon become covered by certain black or brown fungi known as sooty molds, hence interfering with plant photosynthesis.

Included in such plant sap-sucking insects are aphids or Homopteran insects of the Aphididae, and plant sap-sucking insects as used herein include but are not limited to the peach-potato aphid Myzus persicae, the bean aphid Aphis fabae, the pea aphid Acyrthosiphum pisum, the cabbage aphid Brevicoryne brassicae, the grain aphid Sitobion avenae, the rose-grain aphid Metopolophium dirhodum, the Russian wheat aphid Diuraphis noxia (Mordvilko), the English grain aphid Macrosiphum avenae, the greenbug aphid Schizaphis graminum (Rondani), the carrot aphid Cavariella aegopodii, the potato aphid Macrosiphum euphorbiae, the groundnut aphid Aphis craccivora, the cotton aphid Aphis gossypii, the black citrus aphid Toxoptera aurantii, the brown citrus apid Toxoptera ciidius, the willow aphid Cavariella spp., the corn leaf aphid Rhopalosiphum maidis, the aphid Rhopalosiphum padi, the willow leaf aphids Chaitophorus spp., the black pine aphids Cinara spp., the Sycamore Aphid Drepanosiphum platanoides, the Spruce aphids Elatobium spp., Aphis citricola, Lipaphis pserudobrassicae (turnip aphid), Nippolachnus piri, the foxglove aphid Aulacorthum solani, the asparagys aphid Brachycorynella asparagi, the brown ambrosia aphid Uroleucon ambrosiae, the buckthom aphid Aphis nasturtii, the corn root aphid Aphis maidiradicis, the cresentmarked lily aphid Neomyzus circumflexes, the goldenglow aphid Dactynotus rudbeckiae, the honeysuckle and parsnip aphid Hyadaphis foeniculi, the leafcurl plum aphid Brachycaudus helichrysi, the lettuce root aphid Pemphigus bursarius, the mint aphid Ovatus crataegarius, the artichoke aphid Capitophorus elaeagni, the onion aphid Neotoxoptera formosana, the pea aphid Macrosiphum pisi, the rusty plum aphid Hysteroneura setariae, the shallot aphid Myzus ascalonicus, the solanum root aphid Smynthurodes betae, the sugarbeet root aphid Pemphigus betae, the tulip bulb aphid Dysaphis tulipae, the western aster root aphid Aphis armoraciae, the white aster root aphid Prociphilus erigeronensis.

Also included herein as plant sap-sucking insects are whiteflies or Aleyrodidae insects such as Trialeurodes vaporariorum (greenhouse whitefly), the banded wing whitefly Trialeurodes abutilonea, Bemisia tabaci (sweetpotato whitefly) and Bemisia argentifolli (silverleaf whitefly). Also included herein as plant sap-sucking insects are fleahoppers such as Pseudatomoscelis seriatus or cotton fleahopper, and Halticus bractatus or garden fleahopper, Pentatomidae (stink bugs, e.g., Thyanta spp.), mealybugs (Hemiptera, Coccoidea, Pseudococcidae, e.g., the citrus mealy bug (genus Pseudococcus)), as well as Delphacidae (or planthoppers) such as Laodelphax striatellus (small brown planthopper), Nilaparvata lugens (rice brown plant hopper) and Sogatella furcifera (white-backed rice planthopper), and Deltocephalidae (or leafhoppers) such as Flexamia DeLong spp., Nephotettix cincticeps and Nephotettix virescens, Amrasca bigutulla, and the potato leafhopper Empoasca filament. Also included are scales (also named scale insects) such as Aonidiella aurantii (California red scale), Comstockaspis perniciosa (San Jose scale), Unaspis citri (citrus snow scale), Pseudaulacaspis pentagona (white peach scale), Saissetia oleae (brown olive scale), Lepidosaphes beckii (purple scale), Ceroplastes rubens (red wax scale) and Icerya purchasi (cottonycushion scale), besides Tingidae (or lace bugs) and Psyllidae insects, and spittle bugs.

Further included as plant sap-sucking insects are Heteropteran insects and Hemipteran insects of the Auchenorrhyncha that feed from the plants' vascular system, such as sap-sucking insects of the Cicadoidea (such as Cicadas), Cercopoidea (spittlebugs or froghoppers), Membracoidea (leafhoppers and treehoppers), and Fulgoroidea (planthoppers), e.g., the cotton seed sucker bug Dysdercus peruvianus (Heteroptera, Pyrrhocoridae), the apple dimpling bug, Campylomma liebknechti (Hemiptera:Miridae) and the green mirid, Creontiades dilutus which are cotton sucking insect pests, and the Lygus bugs (Hemiptera:Miridae, e.g., Lygus hesperus).

In one embodiment of this invention, phloem-feeding plant sap-sucking insects are targeted with the plants, compositions and methods of the invention, particularly aphids, planthoppers and whiteflies. However, the invention can be applied to any plant sap-sucking pest or any pest which ingests plant sap when feeding on a plant, whether an insect or not, since the mechanism of dsRNA feeding presented herein can similarly affect such plant pest when it ingests dsRNA or siRNA from the plant, e.g., Thrips insects (Thysanoptera, e.g., Thrips tabaci and Frankliniella schultzei). The invention is similarly applicable to other small sucking plant pests, e.g., mites and spider mites (e.g., Tetranychus spp., especially Tetranychus urticae, T. ludeni, T. turkestani, T. pacificus, Tcinnabarinus and T. lambi) sucking on individual plant cells, since they will also ingest and be affected by the dsRNA or siRNA produced in the plant of the invention.

This inventions is similarly applicable to any arthropod pest, insect or otherwise, that feeds on plant cells or tissues and that ingests the dsRNA or siRNA produced in the plants of the invention in a naked, unpackaged form, particularly pests of the Homoptera, Hemiptera and Arachnida pests.

In one embodiment of the current invention, the dsRNA or siRNA chimeric gene construct is present in a plant already expressing an insecticidal protein, e.g., an insecticidal protein derived from Bacillus thuringiensis. Preferred plants expressing such proteins include but are not limited to: corn plants containing the MON863 transformation event, corn plants containing the MON810 transformation event, corn plants containing the Bt11 transformation event, corn plants expressing a Cry1F protein, cotton plants expressing a Cry1Ac protein or cotton plants expressin a Cry1Ac and a Cry2Ab protein (Bollgard™ I or II), cotton plants expressing a Cry1F, Cry1F/1Ac hybrid protein or a VIP3A protein, and corn or cotton plants combining such transgenic events in the same plant species.

Preferred embodiments of the invention are shown in the below Examples which are a selection of possible embodiments and an illustration of the invention. It is evident that a variety of noncritical parameters can be changed to give the same or essentially the same result. All references and published patent applications cited in this application are incorporated herein by reference.

The following sequences are enclosed to this application in the sequence listing:

SEQ ID NO: 1—designed degenerate primer DNA sequence of eIF1A-F primer

SEQ ID NO: 2—designed degenerate primer DNA sequence of eIF1A-R primer (n at position 20 is uncertain and can be a, c, g, or t)

SEQ ID NO: 3—designed degenerate primer DNA sequence of alpha-tubulin-F primer

SEQ ID NO: 4—designed degenerate primer DNA sequence of alpha-tubulin-R primer

SEQ ID NO: 5—DNA sequence of the A. gossypii eIF1A gene PCR fragment

SEQ ID NO: 6—DNA sequence of the M. persicae eIF1A gene PCR fragment

SEQ ID NO: 7—DNA sequence of the A. gossypii alpha-tubulin gene PCR fragment (n at positions 591, 592 and 637 is uncertain and can be a, c, g, or t)

SEQ ID NO: 8—DNA sequence of the M. persicae alpha-tubulin gene PCR fragment (n at positions 3, 113, 128, 137, 509, 615, 617, and 627 is uncertain and can be a, c, g, ort)

SEQ ID NO: 9—designed primer sequence of the Myzus persicae EcR C domain forward primer

SEQ ID NO: 10—designed primer sequence of the Myzus persicae EcR E domain reverse primer

SEQ ID NO: 11—DNA sequence of the Myzus persicae EcR PCR fragment

SEQ ID NO:12—DNA sequence of the Myzus persicae alpha-actinin gene fragment (n at position 704 is uncertain and can be a, c, g or t).

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany. Detailed information and references on RNA interference can be found on the website of Ambion, at: www.ambion.com.

### EXAMPLES

### Example 1

### Injection and Feeding of Aphids with dsRNA Insect Rearing

Aphis gossypii were raised on 3-5 week old cotton plants, at 25° C. under a 14 hour:10 hour day/night cycle, with low to medium humidity. Between 5 and 10 apterae adults were placed on each plant on a weekly basis. Myzus persicae were raised on 3-5 week old radish plants, at 20° C. under a 14 hour:10 hour day/night cycle with low to medium humidity. Between 10 and 15 apterae adults were placed on each plant every two weeks.

PCR Amplification and Sequence Analysis of Aphid Genes

Total RNA was extracted from a population of aphids of mixed life stages using a Qiagen RNeasy Minikit™, according to the manufacturer's specifications. The RNA (−250 ng) was used to prepare cDNA using Invitrogen's Thermoscript RT-PCR System™, using oligo(dT) primers. The resulting cDNA was then used as template for the amplification of the eukaryotic initiation factor.

Degenerate PCR primers were designed to span moderate to highly conserved regions of the eukaryotic initiation factor 1A (eIF1A) gene and the alpha-tubulin genes, based on alignments of multiple sequences of invertebrate's genes derived from GenBank. Sequences were aligned and multiple sequence comparisons were generated using either the GCG program ‘Pileup’ or ‘CLUSTAL W’ with default parameters for the nucleotide sequences and the default-scoring matrix for proteins. Primers were designed to cover a single putative exon whenever possible. Exon predictions were usually based on exon boundaries found in the Drosophila melanogaster orthologue of the target gene. To assist with the design process, the CODEHOP program (Blocks Server, http://www.blocks.fhcrc.org) was used.

TABLE 1

PCR primers used to isolate gene
sequences from A. gossypii and M. persicae

| | |
|---|---|
| Primers | Primer sequence |

| | |
|---|---|
| eIF 1A primers: | |
| EIF1A-F (SEQ ID NO:1) | 5′ AAA ACA GAA GAA GAG |
| | GTA AAA AYG ARA 3′ |
| EIF1A-R (SEQ ID NO:2) | 5′ GGT TTC TGG CTT CGT |
| | CTG GNG TRT AYT T 3′ |
| Alpha-tubulin primers: | |
| Tubulin-F (SEQ ID NO:3) | 5′ TAC AAC TCS ATC YTG |
| | ACC AC 3′ |
| Tubulin-R (SEQ ID NO:4) | 5′ TCC ATR CCY TCW CCB |
| | ACR TAC C 3′ |

Amplifications of the target genes were performed using Invitrogen's Thermoscript RT-PCR System, with Platinum Taq DNA polymerase, on a Corbett Research Thermocycler. A range of annealing temperatures (0.5 to 5 degrees below the predicted Tₘ of the primers), and a range of MgCl₂ concentrations (0 to 3.0 mM) were tested to find the conditions that produced the maximum amount of specific PCR product. The following PCR conditions were used: 94° C. for 2 min, 30 cycles of [94° C. for 30 sec; 47-52° C. for 30 sec; 72° C. for 90 sec], and 72° C. for 3 min.

The PCR products were gel purified using a Perfectprep Gel Cleanup Kit (EppendorF) and subcloned into the plasmid pGEM-T-Easy (Promega). Isolated plasmid DNA was sequenced using primers complementary to the T7 and SP6 promoters in the vector. DNA was sequenced using BigDye-terminator chemistry (Applied BioSystems) according to the manufacturer's specifications, and sequencing reactions were resolved and processed by an Applied BioSystems Model377XL automated DNA sequencer.

DNA sequences were edited to remove plasmid sequences and pairwise comparisons were performed using the GCG alignment program ‘Gap’ (Devereux et al., 1984) and multiple sequence comparisons and consensus sequences were generated using either the GCG program ‘Pileup’ (Devereux et al., 1984) or ‘CLUSTALW’ (Thompson et al., 1994) with the default parameters (gap weight 5.0, gap length weight 0.3) for nucleotide sequences.

A single 279 nt long PCR product was amplified from A. gossypii and M. persicae cDNA using the aforementioned eIF primers. Each PCR product was sequenced, and based on DNA sequence comparisons with other known invertebrate eIF genes, both were confirmed to be eIF gene fragments. SEQ ID NO: 5 and 6 show the DNA sequences of the A. gossypii and M. persicae eIF1A PCR fragments, respectively.

The sequences of the alpha-tubulin A. gossypii and M. persicae PCR fragments corresponding to the genes found in the cDNA library of these insects is shown in SEQ ID NO: 7 and 8, respectively.

Using the above protocol, also DNA sequences for the A. gossypii alpha-actinin, acetylcholinesterase and elongation factor 1-alpha gene are determined, based on the available sequences of the Drosophila orthologs of these gene in Genbank accession numbers NM058137 (alpha-actinin), X05893 (acetylcholinesterase), or based on the available sequence of the numbers M. Persicae ortholog of the elongation factor 1 alpha gene in Genbank accession number AF143612.

Using a similar procedure as described above for the eIF and alpha-tubulin genes, also a DNA sequence of a Myzus persicae alpha-actinin gene was isolated. This sequence is shown in SEQ ID NO:12.

Preparation of Double-Stranded RNA

To facilitate in vitro dsRNA synthesis, the gene fragment from the pGEM-T-Easy plasmid was subcloned into the pL4440 plasmid (Timmons and Fire, 1998), a plasmid with two convergent T7 promoters. Transcription from this plasmid requires the use of only T7 RNA polymerase to produce both sense and antisense RNAs simultaneously, which will anneal in vitro to form dsRNA.

The plasmid pL4417 (described in Fire Lab 1997 Vector Supplement, February 1997, provided by Andrew Fire, Carnegie Institute; Timmons et al., 2001) contains the Aequorea victoria green fluorescent protein gene, gfp, flanked by two convergent T7 promoters. This plasmid was used to prepare dsRNA of the entire (716 nt) open reading frame of the gfp gene, to assess whether non-aphid specific dsRNA had any deleterious effects on the aphids.

To obtain sufficient DNA template for in vitro transcriptions, the aphid gene fragment and flanking T7 promoter sequences were PCR-amplified from the pL4440 plasmid using plasmid-specific primers.

Double-stranded RNA was prepared using a T7 RiboMAX Express Large Scale RNA production System (Promega) according to the manufacturer's specifications.

Insect Bioassays

To test if the A. gossypii eIF1A dsRNA was functional in silencing the aphid gene, haemocoel injections of dsRNA targeting this aphid gene were done in A. gossypii adult females and the mortality in time and the fecundity was compared to control, buffer-injected or dsGFP (green fluorescent protein)-injected A. gossypii. 25 apterae adults were injected with 1 microg/microl dsRNA in injection buffer (5 mM KCl, 0.1 M sodium phosphate, pH 6.8). Ten apterae adults were similarly injected with dsGFP (1 microg/microl) in injection buffer and 10 apterae adults were injected with injection buffer only. Injected aphids were placed individually in arenas containing the artificial aphid diet described below. Mortality of the adults and the number of live nymphs were recorded at 3, 7, 11, and 14 days post injection, after which the experiment was terminated. This regime was repeated three times such that a total of 75 adults had been injected with the aphid-specific dsRNA. Injection of eIF dsRNA resulted in a considerable percentage (29%) of A. gossypii adults dying within 3 days following the injection, and the remaining injected aphids continued to die over the following 2 weeks. In comparison, considerable fewer aphids died at 3 days post injection when injected with either GFP dsRNA (3%) or injection buffer (7%).

Injections of M. persicae with M. persicae eIF dsRNA also killed some aphids, but the mortality rates in this experiment did not appear to be significant when compared to GFP dsRNA- or buffer-injected controls (a large variation was observed in the controls and the treatment).

For those A. gossypii females that survived the injection treatment, their nymph production was assessed over a two week period. The cumulative number of nymphs produced at 7, 11, and 14 days post injection were similar for all treatments, with the aphid-specific dsRNA having no significant effect on the reproduction of the treated females that survived.

Similarly, a portion of the Myzus persicae ecdysone receptor (EcR) gene was amplified from M. persicae cDNA using the following PCR primer sequences:

| | |
|---|---|
| MpEcR C domain forward | |
| primer: | |
| 5′ CCCAAGCTTTGCCTGGTGTGTGGC | (SEQ ID NO:9) |
| GACCGG 3′. | |
| MpEcR E domain reverse | |
| primer: | |
| 5′ CCCAAGCTTATCCTGGAAATAGAC | (SEQ ID NO:10) |
| AAGTCG 3′. | |

The underlined adapters were added to provide a HindIII site for subcloning into a dsRNA expression vector.

The about 450 bp PCR product was gel-purified using a Perfectprep Gel Cleanup Kit (Eppendorf) and subcloned into the plasmid pGEM-T-Easy (Promega). The gene fragments from the pGEM-T-Easy plasmid were subcloned into the pL4440 plasmid (kindly provided by Andrew Fire), a plasmid with two convergent T7 promoters. Transcription from this plasmid requires the use of only T7 RNA polymerase to produce both sense and antisense RNAs simultaneously, which will anneal in vitro to form dsRNA. Double-stranded RNA was prepared using a T7 RiboMAX Express Large Scale RNA production System (Promega) according to the manufacturer's specifications.

SEQ ID NO:11 shows the Myzus persicae ecdysone receptor DNA sequence coding from the start of the C domain to the 15th residue of the E domain (about 450 bp).

Two to three day old adult females were microinjected into their abdominal haemocoels with 50 nL of buffer, with or without 1 ug/uL dsRNA (eIF1A or EcR). Aphids were anaesthetised using CO2, and secured to a microscope slide coated with a dried, sticky layer of 20% sucrose. The insects were injected using borosilicate glass needles, prepared using a micropipette puller (Sutter Instruments) and sometimes sharpened with a needle beveller (Narashige). The needle was operated using a micromanipulator (Narashige) secured to a stereomicroscope, and the fluid was delivered with the aid of an air-driven pump controlled by a foot-operated solenoid switch. Injected aphids were washed from the sucrose pad, and transferred to individual feeding arenas. The arenas consisted of small (1.5 cm inner diameter) plastic cylinders closed at one end with a glass microscope coverslip and at the other end with two Parafilm membranes. An artificial liquid diet (see below) was contained between the two Parafilm membranes, and the survival of the injected aphids, and the number of nymphs that they produced over a 10-day period was monitored.

No increase in aphid mortality was observed for M. persicae aphids injected with EcR dsRNA. Aphids injected with buffer only or with eIF dsRNA produced a similar number of nymphs at days 5 and 10 post injection (FIG. 1). Aphids injected with EcR dsRNA produced significantly fewer nymphs after day 10 (n=25, Student's T-test, P<0.01), suggesting that although the injected dsRNA had taken some time to promote an effect on nymph production, it adversely affected insect fecundity.

[image]

Also, injection of Aphis gossypii alpha-tubulin dsRNA in the haemocoel of these aphids shows that aphid mortality is increased compared to control dsGFP injections. Hence, the A. gossypii tubulin sequence of SEQ ID NO: 7 or the gene corresponding to this sequence is another possible target gene.

For the feeding assays, three to four adult female A. gossypii aphids were placed in individual feeding arenas and left for 24 h. The adults were then removed, leaving the newborn nymphs to develop entirely on the artificial diets containing dsRNA. The arenas consisted of small (1.5 cm inner diameter) plastic cylinders closed at one end with a glass microscope coverslip or plastic film and at the other end with two Parafilm membranes. A total of 100 microliter of artificial liquid diet was contained between the two Parafilm membranes, containing either 1 microgram/microliter dsRNA (gfp or eIF) or 10 microliter RNA dilution buffer (10 mM Tris, pH 8.0). Three to five independent preparations of eIF and gfp dsRNA were pooled to provide sufficient quantities of dsRNA (5 mg) for the feeding bioassays. The artificial diet was prepared as a 2× stock, so that following addition of dsRNA and buffer, the final concentration of diet could be diluted to 1× concentration by adding the appropriate volume of water. Between 45 and 50 arenas were set up for each treatment (buffer, gfp-dsRNA, and eIF-dsRNA), starting with, on average, 40 nymphs per arena at day 1. The number of live aphids at days 3, 7, and 11 were recorded, and the percentage of aphids surviving for each arena, relative to day 1, was calculated.

The artificial diet used includes, for 9 ml (all ingredients from cell culture quality): amino acid stock solution (1 ml), vitamin stock solution (0.3 ml), sucrose 80% (3.125 ml), potassium-sodium phosphate buffer (1 ml), ovalbumin (10 mg), MgCl₂ (10 mg), Wesson salts (10 mg), water (3.57 ml). The potassium-sodium buffer solution (100 mM, pH 7.0) contains: NaH₂PO₄: 1.4 g, K₂HPO₄: 2.6 g, Ascorbic Acid: 0.25 g, adjust pH to 7.0, mix with 250 ml water (this is not stored, an amount is made to use immediately in the diet which is stored). The diet is mixed, filtered on 0.2 μm filters and stored in 5 ml tubes at −70° C. A large amount is prepared, which is stored in the freezer for ±6 months. Just before a bioassay, antibiotics are added: to 9 ml of diet, 20 μl erythromycine (10 mg/ml EtOH), 10 μl triacilline (100 mg/ml), 5 μl chloramphenicol (60 mg/ml), 6 μl kanamycine (50 mg/ml). This has some negative effect on growth of larvae and can be omitted when working with cell-free samples.

The amino acid stock solution contains (all L-amino acids, between brackets final concentration in mM are indicated): Glutamine (32.8), Serine (25.5), Arginine (10.7), Valine (7.1), Threonine (6.9), Leucine (5.1), Lysine (5.1), Isoleucine (4.4), Phenylalanine (2.8), Histidine (2.2), Methionine (1.6), Alanine (1.6), Tryptophan (0.9).

The vitamin stock solution contains (between brackets mg/10 ml): biotin (0.1), Ca-pathotenate (5), choline chloride (50), myo-inositol (50), niacin (10), pyridoxin (2.5), thiamine-HCl (2.5).

The amino acid stock solution and the vitamin stock solution are not stored: an amount is prepared to use immediately in the diet which is stored.

Feeding eIF1A dsRNA to A. gossypii nymphs resulted in a significant reduction in the survival of nymphs (FIG. 2). After 11 days, only 17 (±1.4) % of the aphids fed on diet containing eIF dsRNA had survived, whereas 54 (±4.1) and 51 (±2.8) % of the aphids fed on diet containing buffer or gfp dsRNA had survived. Hence, there is a two-fold increase in aphid mortality due to the presence of the eIF dsRNA in the diet. Student t-tests confirmed that the feeding of eIF dsRNA significantly reduced the survivorship of aphids, relative to aphids fed either buffer or gfp dsRNA (P<0.0001).

[image]

These data of a significantly higher mortality of aphids when fed dsRNA have been confirmed in another experiment wherein a smaller number of aphids was used, but using a similar experimental setup as above (also using the A. gossypii eIF1A dsRNA fed to A. gossypii nymphs).

A similar experimental setup with M. persicae aphids and a dsRNA based on the above M. persicae eIF1A sequence (SEQ ID NO:6) confirms that a significant mortality is obtained when feeding these aphids naked, unpackaged dsRNA molecules in their diet.

These results show that surprisingly, significant mortality is found in aphids fed naked, unpackaged dsRNA targeted to an essential aphid gene.

Also, insect mortality and efficient silencing is obtained by using small interfering RNA molecules (siRNA) directly in the aphid diet. For 3 target genes (eIF, tubulin and GFP), siRNA is prepared by long dsRNA cleavage using the RNAseIII (Ambion's Silencer siRNA Cocktail Kit, generating 12-30 bp siRNAs) or recombinant human dicer (Gene Therapy Systems, producing (more optimal) 21-23 bp siRNAs). Aphids are fed artificial diet containing a concentration of long dsRNA and siRNAs to determine the difference in efficacy. Application of siRNA molecules in the aphid diet also produces a significant effect on aphid development, comparable to or even better then the longer dsRNA applied above.

Addition of a poly-Arginine 12-mer cationic oligopeptide to the aphid diet, together with the eIF1A dsRNA, results in a significantly increased mortality in the above feeding assays towards A. gossypii and M. persicae, compared to the application of dsRNA without such peptide and a control setup using GFP dsRNA.

### Example 2

### Analysis of Aphid Feeding on Plants Transiently Expressing dsRNA

To confirm that plants can deliver dsRNA or siRNA to plant sap-sucking insects such as aphids in planta without transfection promoting agents, aphids are added to tobacco plants transiently induced to produce aphid dsRNA using the SVISS methodology described in published PCT application WO 03/052108 (incorporated herein by reference), using dsRNA targeted to a M. persicae essential gene.

These experiments confirm that the dsRNA transiently produced in plants in an unpackaged form, free from transfection-promoting agents, can result in a significant inhibition of M. persicae development.

### Example 3

### Analysis of Aphid Feeding on Plants Stably Expressing dsRNA in Phloem

In this experiment, Arabidopsis and tobacco plants are transformed by Agrobacterium-mediated transformation with a vector containing a dsRNA chimeric gene targeting either the M. persicae eIF1A gene, the M. persicae ecdyson receptor (EcR) or the control GFP gene. A M. persicae ecdyson receptor DNA sequence was cloned from this aphid as described above. A plant dsRNA chimeric gene is made wherein either the phloem-specific rolC promoter (Pandolfini et al., 2003) or the constitutive CaMV 35S promoter is operably linked to the dsRNA construct, containing sense and antisense regions to the above target aphid gene or the control gene. Plants are tested for transformation using Southern blot analysis and for expression and dicing of the dsRNA by small RNA Northern blot assay. Transport of interfering RNAs to the phloem is confirmed by the significant reduction in development of Myzus persicae feeding on the successfully transformed Arabidopsis and tobacco plants. Northern blot analysis confirms the presence of the RNA molecules of the invention in the phloem sap of plants that are successfully transformed.

Hence, plant sap-sucking insects can now be controlled using specific and selective dsRNA-sequences targeting essential plant sap-sucking genes so as to minimize population build-up of aphids on crop plants.

### CITED REFERENCES

Adams and Sekelsky (2002) Nature Reviews Genetics 3:189.
Almon et al. (1997) Plant Physiol. 115:1599-1607.
An et al. (1996) The Plant J. 10, 107.
Ashford et al. (2000) J. Ins. Physiol. 46:335-341.
Bender et al. (1997) Cell. 91:777-88.
Brears et al. (1991) Plant J. 1:235-244.
Ceci et al. (2003) The Plant J. 33:557-566.
Christensen et al. (1992) Plant Mol. Biol. 18, 675-689.
Christou et al. (1990) Trends Biotechnology 8, 145.
Clarkson and Saint (1999) DNA Cell. Biol. 18:457-62.
Clarkson et al. (1999) Nature 399:694-7.
Cornejo et al. (1993) Plant Mol. Biol. 23, 567-581.
Cristofoletti et al. (2003) J. Ins. Physiol. 49:11-24.
Datta et al. (1990) Bio/Technology 8, 736-740
Dehio et al. (1993) Plant Mol. Biol. 23:1199-1210.
de Pater et al. (1992) The Plant J. 2, 834-844.
Depicker et al. (1982) J. Molec. Appl. Genetics 1, 561-573.
Devereux J, et al. (1984) Nucleic Acids Res. 12:387-395.
Down et al. (1996) J. Insect Physiol. 42:1035-1045.
Dubreuil and Wang (2000) J. Muscle Res. Cell Motil. 21:705-13.
Edwards et al. (1990) Proc. Natl. Acad. Sci. USA 87:3459-3463.
Foissac et al. (2002) Insect Biochem. Mol. Biol. 32:967-978.
Franck et al. (1980) Cell 21, 285-294
Fromm et al. (1990) Bio/Technology 8, 833-839
Funk et al. (2002) Plant Physiol. 128:84-94.
Fyrberg et al. (1998). Biochem. Genet. 36:299-310
Gardner et al. (1981) Nucleic Acids Research 9, 2871-2887
Gielen et al. (1984) EMBO J. 3:835-845
Gordon-Kamm et al. (1990) The Plant Cell 2, 603-618
Graham et al. (1997). Plant Mol. Biol. 33:729-735.
Guivarc'h et al. (1996). Transgenic research 5:3-11.
Gura (2003) Nature 404:804-808.
Hay (2000) Cell. Death Differ. 7:1045-56.
Hedley et al. (2000) J. Exp. Botany 51:817-821.
Hehn and Rohde (1998). J. Gen. Virol. 79:1495-1499.
Henrich et al. (2000) Genesis 28:125-33.
Hinchee et al. (1988) Bio/Technology 6, 915.
Hovemann et al. (1988) Nucl. Acids Res. 16:3175-94.
Hull and Howell (1987) Virology 86, 482493.
Ito et al. (2000) Plant Science 155:85-100.
Jarver and Langel (2004) Drug Discovery Today 9:395-402.
Jiang et al. (2000). Dev. Biol. 226:118-36.
Johnson et al. (1998) Gene 221:127-34.
Kertbundit et al. (1991) Proc. Natl. Acad. Sci. USA 88: 5212-5216.
Kiyokawa et al. (1994) Plant Physiology 104:801-802.
Korber et al. (1991) EMBO J. 10:3983-91.
Kuhn et al. (1997) Science 275:1298-1300.
Lam and Thummel (2000) Curr. Biol. 10:957-63.
Last et al. (1990) Theor. Appl. Genet. 81, 581-588.
Leyva et al. (1992) Plant Cell. 4: 263-271.
Magrelli et al. (1994) Science 266:1986-1988.
Margolis and Duyk (1998) Nature Biotech. 16:311.
Marzoui et al. (1999) RNA 5:1615-31.
Matthews and Kaufman (1987) Dev. Biol. 119:100-114
Medberry et al. (1992) Plant Cell 4:185-192.
Moran and Thompson (2001) Plant Physiol. 125:1074-1085.
Mottus et al. (2000) Genetics 154:657-68.
Myrick and Dearolf (2000) Gene 244:119-125.
Needleman and Wunsch (1970) J. Mol. Biol. 48:443-53.
Newmark et al. (2003) P.N.A.S. USA 100, suppl. 1, 11861-11865.
Nolte and Koch (1993) Plant Physiol. 101:899-905.
Pandolfini et al. (2003) BioMedCentral (BMC) Biotechnology 3:7 (http://www.biomedcentral.com/1472-6750/3/7)
Pathirana et al. (1997). Plant J. 12:293-304.
Rahbe et al. (1995) Entomologia Experimentalis et Applicata 76:143-155.
Rajagopal et al. (2002). J. Biol. Chem. 277:46849-46851.
Ramloch-Lorenz et al. (1993). The Plant J. 4:545-554.
Rao et al. (1998) The Plant Journal 15:469-477.
Rohde et al. (1994) Plant Mol. Biol. 27:623-628.
Rossi et al. (1998) Proc Natl. Acad. Sci. USA 95:9750-9754.
Roy et al. (2002) J. Agric. Food Chem. 50:6775-9.
Schunmann et al. (2003) Plant Functional Biology 30:453-460.
Shi et al. (1994) J. Exp. Bot. 45:623-631.
Shigenobu et al. (2000) Nature 407:81-86.
Shimamoto et al. (1989) Nature 338, 274-276.
Smith et al. (2000) Nature 407:319-20.
Spena and Langenkemper (1997) Genet. Res. 69:11-15.
Spradling et al. (1999) Genetics 153:135-177.
Stoger et al. (1999) Molecular Breeding 5:65-73.
Thompson et al. (1994) Nucleic Acids Res. 22:4673-4680.
Timmons et al. (2001) Gene 263,103-112.
Timmons and Fire (1998) Nature 395:854.
Tomero et al. (1996) Plant J. 9:639-648.
Torres-Schumann et al. (1996) Plant J. 9:283-96.
Tortiglione et al. (2003) Plant Mol Biol. 53(6):891-902.
Unnamalai et al. (2004) FEBS Letters 566:307-310.
van Daal and Elgin (1992) Mol Biol Cell 3:593-602.
Velten and Schell (1985) Nucleic Acids Research 13, 6981-6998 (1985)
Velten et al. (1984) EMBO J 3, 2723-2730 (1984).
Verdaguer et al. (1998) Plant Mol. Biol. 37, 1055-1067 (1998).
Wu et al. (2000) Plant J. 22:495-502.
Yin and Beachy (1995) Plant J. 7:969-980.
Yin et al. (1997) Plant J. 12:1179-88.
Yingqiu et al. (2000) Science in China (Series C) vol. 43:498-506.
Yoshimoto et al. (2003) Plant Physiol. 131:1511-1517.
Zeremski et al. (2003) Genesis 35:31-38.
Zhang et al. (1991) The Plant Cell 3, 1155-1165.
Zhang et al. (1996) Plant Physiol. 112:1111-1117.
Zhou et al. (1998) Chin. J. Biotechnol. 14:9-16.

1. A chimeric gene comprising the following operably linked DNA:
(a) a plant-expressible promoter;
(b) a DNA region which when transcribed yields a double-stranded RNA molecule capable of reducing the expression of an essential gene of a plant sap-sucking insect, said RNA molecule comprising a first and second RNA region wherein:
(i) said first RNA region comprises a nucleotide sequence of at least 19 consecutive nucleotides having at least about 94% sequence identity to the nucleotide sequence of said endogenous gene;
(ii) said second RNA region comprises a nucleotide sequence complementary to said at least 19 consecutive nucleotides of said first RNA region;
(iii) said first and second RNA region are capable of base-pairing to form a double stranded RNA molecule between at least said 19 consecutive nucleotides of said first and second region;
(c) optionally, a 3′ end region comprising transcription termination and polyadenylation signals functioning in cells of said plant.

2. The chimeric gene of claim 1, wherein said essential of said plant sap-sucking insect is selected from the group consisting of genes encoding the following: a gut cell protein, a membrane protein, an ecdyson receptor, a γATPase, an amino acid transporter, a transcription factor, a peptidylglycine alpha-amidating monooxygenase; a cystein protease, an aminopeptidase, a dipeptidase, a sucrase/transglucosidase, a translation elongation factor, an eucaryotic translation initiation factor 1A, a splicing factor, an apoptosis inhibitor; a tubulin protein, an actin protein, an alpha-actinin protein, a histone, a histone deacetylase, a cell cycle regulatory protein, a cellular respiratory protein; a receptor for an insect-specific hormonal signal, a juvenile hormone receptor, an insect peptidic hormone receptor; a protein regulating ion balance in a cell, a proton-pump, a Na/K pump, an intestinal protease; an enzyme involved in sucrose metabolism, a digestive enzyme, a trypsin-like protease and a cathepsin B-like protease.

3. The chimeric gene of claim 1, wherein said double-stranded RNA silences the gene corresponding to the DNA sequence of any one of SEQ ID NO: 5 to 8, SEQ ID NO: 11 or SEQ ID NO: 12.

4. The chimeric gene of claim 1, wherein between said first and second RNA region, a spacer region containing a plant intron is present.

5. The chimeric gene of claim 1, wherein said essential gene has a portion which occurs with the same sequence or with at least 94% sequence identity in homologous genes of several plant sap-sucking insects.

6. The chimeric gene of claim 1, wherein said promoter is a constitutive promoter.

7. The chimeric gene of claim 1, wherein said promoter is a vascular-specific or a phloem-specific promoter.

8. The chimeric gene of claim 7, wherein vascular- or phloem-specific promoter is selected from the group consisting of: a rolC or rolA promoter of Agrobacterium rhizogenes, a promoter of a Agrobacterium tumefaciens T-DNA gene 5, the rice sucrose synthase RSs1 gene promoter, a Commelina yellow mottle badnavirus promoter, a coconut foliar decay virus promoter, a rice tungro bacilliform virus promoter, the promoter of a pea glutamine synthase GS3A gene, a invCD111 and invCD141 promoters of a potato invertase genes, a promoter isolated from Arabidopsis shown to have phloem-specific expression in tobacco by Kertbundit et al (1991), a VAHOX1 promoter region, a pea cell wall invertase gene promoter, an acid invertase gene promoter from carrot, a promoter of a sulfate transporter gene Sultr1;3, a promoter of a plant sucrose synthase gene, a promoter of a plant sucrose transporter gene.

9. A plant cell, tissue, or a plant or a plant seed comprising the chimeric gene or the double-stranded RNA molecule described in claim 1.

10. A method to silence a gene of a plant sap-sucking insect, comprising applying to the feed of said plant sap-sucking insect unpackaged, naked dsRNA or siRNA which is targeted to an essential plant sap-sucking gene.

11. The method of claim 10, wherein said essential gene is any of the genes listed in claim 2 above.

12. The method of claim 10, wherein said application is by expression of a dsRNA chimeric gene in phloem cells of a plant.

13. A method to silence a gene in an plant sap-sucking insect, comprising: adding naked, unpackaged dsRNA or siRNA to the diet or feed of said plant sap-sucking insect, wherein said dsRNA or siRNA targets said gene.

14. A method of controlling plant sap-sucking insects, comprising expressing in the phloem of a plant dsRNA that targets an essential plant sap-sucking insect gene.

15. The method of claim 14 wherein said gene is a gene expressed at least in the intestine or in gut cells.

16. The method of claim 14 wherein said plant sap-sucking insect is an aphid or a whitefly.

17. A plant, comprising stably inserted in its genome, the chimeric gene of claim 1, so that said chimeric gene is expressed in the phloem or xylem of said plant.

18. A method of identifying gene function in a plant sap-sucking insect, comprising the step of applying naked, unpackaged dsRNA targeting a plant sap-sucking insect gene to the diet of said insect, and evaluating phenotypic or biochemical changes in said insect.

19. A method of identification of novel targets for insecticidal compounds, comprising the steps of: a) applying naked, unpackaged dsRNA or siRNA molecules to the feed or diet of a plant-sap sucking insect; b) analyzing which genes when silenced confer lethality to said insect, c) cloning and characterizing said genes thus analyzed; d) identifying compounds that disrupt or inactivate said gene or the RNA or protein encoded thereby; and e) contacting said compounds with said insect or feed or diet of said insect to confirm the pesticidal nature of said compound.

20. Phloem of a plant, comprising siRNA targeted to an aphid essential gene.

21. Phloem sap of a plant, comprising siRNA targeted to an aphid essential gene.

22. An aphid gene comprising the sequence of any one of SEQ ID NO:5 to 8, SEQ ID NO: 11 or SEQ ID NO: 12.

23. The method of claim 18, wherein a cationic oligopeptide is mixed in the diet together with the dsRNA.

24. The method of claim 23, wherein said oligopeptide is a 12 amino acids poly-Arginine peptide.

25. The plant cell, tissue, plant or plant seed of claim 9, which also comprises a chimeric gene encoding a cationic oligopeptide.

26. The plant cell, tissue, plant or plant seed of claim 25, wherein said oligopeptide is a 12 amino acids poly-Arginine peptide.

27. The method of claim 19, wherein a cationic oligopeptide is mixed in the diet together with the dsRNA.

28. The plant cell, tissue, plant or plant seed of claim 17, which also comprises a chimeric gene encoding a cationic oligopeptide.


SEQUENCE LISTING (12 sequences)

SEQ ID NO: 1 - DNA, 27 bp, artificial
  designed degenerate primer
aaaacagaagaagaggtaaaaaygara

SEQ ID NO: 2 - DNA, 28 bp, artificial
  designed degenerate primer
ggtttctggcttcgtctggngtrtaytt

SEQ ID NO: 3 - DNA, 20 bp, artificial
  designed degenerate primer
tacaactcsatcytgaccac

SEQ ID NO: 4 - DNA, 22 bp, artificial
  designed degenerate primer
tccatrccytcwccbacrtacc

SEQ ID NO: 5 - DNA, 279 bp, Aphis gossypii
aaaacagaagaagaggtaaaaatgagaatgaaacagaaaagcgtgagttggtgttcaaagaagatggacaagaatatgctcaagttaccaaaatgttgggaaatggacgtctagaagcaatgtgttttgatggtgtaagacgactttgtcacattcgaggaaaacttaggaaaaaggtgtggatcaatcaagctgacatagtattgataggcttacgtgaatatcaagatacaaaagccgatgtaattttgaaatacaccccagacgaagccagaaacc

SEQ ID NO: 6 - DNA, 279 bp, Myzus persicae
aaaacagaagaagaggtaaaaacgaaaatgaaaccgagaagcgtgaattggtgttcaaagaagatggccaagaatatgctcaagttaccaaaatgttgggaaatggacgtctagaagctatgtgctttgatggtgttaaacgactttgccacatacgaggaaaacttaggaaaaaggtatggattaatcaagctgatatagtattaataggtttacgtgaataccaagacacaaaagccgatgtaattttgaaatacacaccagacgaagccagaaacc

SEQ ID NO: 7 - DNA, 638 bp, Aphis gossypii
  misc_feature: n at 591, 592 and 637 is a, c, g or t
tcatggctggactacgaggccatctacgacatctgccgccggaacctggacatcgagcggcccacgtacacgaacctcaaccggctgatcgggcagatcgtgtcgttcccatcacgggcgtcgctgcggttcgacggcgcgctgaacgtcgacctgaccgagttccagacgaacctggtgccgtacccgcgcattcacttcccgctggccacgtacgcgccggtcatatcggccgagaaggcgtaccacgagcagctgtccgtggccgaaatcaacccaacgcgtgcttcgaaccggccaaaccagaatgggtcaagatgcgacccgcggcacggcaagtacatgggcctgctgcaatgctgtaaccgcggcgacgtcgtgcccaaggacatgaacgcggccatcgccaccatcaagaccaagaggaccatcgtgtacgtcgactggtgcccgaccgggttcaaggtgggcatctactaccagccgccgaccgtggtgccgggggcgatctggccaaggtgcagcgggcggtgtgcatgttgtccaacacgacggccatctccgaggcgtgggcccggctcgaccacaagnntgacctgatgtacgctgacacgcgcgtccgtccactggtacgtang

SEQ ID NO: 8 - DNA, 628 bp, Myzus persicae
  misc_feature: n at 3, 113, 128, 137, 509, 615, 617, and 627 is a, c, g, or t
tgnacacagtgactgtgcattcatggtcgataatgaagccatctatgacatctgccgtcgtaatctcgatattgaacgtgcccacttacactaacttgaatcgtcttattggncagattgtgtcttcnatcacagcntctctccgtttcgatggtgccctcaatgttgacttgactgaattccagaccaatttggtcccatacccccgtattcatttcccattggtcacttatgcaccagtcatctccgctgaaaaggcttaccatgaacaattgtccgtatcagaaatcactaacgcttgttttgaaccagccaaccaaatggtgaaatgtgatccacgtcatggcaaatacatggcttgttgcatgttgtaccgtggtgatgttgtacccaaagacgtcaacgctgccattgcttccatcaagaccaagagaacattcagtttgttgactggtgtccaactggtttcaaagttgggtatcaactaccaacccccaaccgtggtaccnggtgtgacttggtctaaagtacaacgtgctgtctgcatgttgtccaacactacagctattgctgaagcttgggtctaggtttggtaccacaagttcgtaacttgcnantacgtccacna

SEQ ID NO: 9 - DNA, 30 bp, artificial
  designed primer sequence
cccaagctttgcctggtgtgtggcgaccgg

SEQ ID NO: 10 - DNA, 30 bp, artificial
  designed primer sequence
cccaagcttatcctggaaatagacaagtcg

SEQ ID NO: 11 - DNA, 408 bp, Myzus persicae
tcgtccggttaccattacaacgctctcacatgcgaaggatgcaaggggttcttccggaggagcatcaccaagaacgccgtgtaccagtgcaagtacggcaacaattgcgaaatcgacatgtacatgaggcggaagtgccaggagtgccggctgaaaaaatgcctgaccgtcggcatgaggcctgaatgtgttgtacctgaagttcaatgcgcagtaaaaagaaaggagaaaaaagctcaacgagaaaaagataaaccaaattctactacagacatttctcctgaaataataaaaatagaacctacagagatgaagattgaatgtggtgaaccaatgataatgggcacacctatgccgactgtaccttacgtgaaacctttgagttctgaacaaaaagaactgatccac

SEQ ID NO: 12 - DNA, 1173 bp, Myzus persicae
  misc_feature: n at 704 is c, g, a, or t
tgccagcgcatttgcgaccagtgggacagattaggtagcttgacacagaaacggagaactgacttggatgatgcagaaaaaatattagagaaaattgatatattgcatttggaattcgctaagagagcagctcctttcaacaactggttggatggtacacgtgaagatttagtggacatgttcattgtacacactgttgaggaaatccaaggattgattgatgcacatggacaatttaaggctactttgtctgatgctgacaaagagtacaactctatcattggactggtcaaagatgttgagtcaactgtacaaaaataccaaatacctggtggtcttcagaacccgtacactactttgacttctagtgatttaagcaaaaaatggtctgaagtgaaacatttagtgccccaaagagacacgaccctccaagctgaactcagaaaacaacaaaacaatgagatgctacgtcgtcaatttgcggagaagtcaaatcaagtgggtccttggattgagaggcaaatggacgctgtcacggccatcggtatgggattgcaggttctctggaagatcaattgcaccaactgaaacaatttagggctactttgtctgatgctgacaagagtacaactctatcattggactggtcaagatgttgagtcaactgtacaaaaataccaaatacctggtggtcttcagaaccngtacactactttgacttctagtgatttaagcaaaaaatggtctgaagtgaaacatttagtgccccaaagagacacgaccctccaagctgaactcagaaaacaacaaaacaatgagatgctacgtcgtcaatttgcggagaagtcaaatcaagtgggtccttggattgagaggcaaatggacgctgtcacggccatcggtatgggattgcaaggttctctggaagatcaattgcaccaactgaaacaatacgaacagaatgtgtttgcatacaagccacatattgaggaattagagaaaatccaccaagctgtacaagagggtatgatcttcgaaaacaggtatactcaatacacaatggagacattacgtgttggatgggaacaactattgacgtccataaatcgcaatgtgaatgaagtagaaaaccaaatattgaccagagactccaaaggcatcacccaggagcag